(19) United States
(12) Patent Application Publication
(10) Pub. No.: US 2026/0076605 A1
(43) Pub. Date: Mar. 19, 2026

(54) CARD CAPABLE OF MEASURING ECG SIGNALS

(71) Applicant: ANICA CORPORATION, Taoyuan City (TW)

(72) Inventor: MENG-JEN CHENG, New Taipei City (TW)

(73) Assignee: ANICA CORPORATION, Taoyuan City (TW)

(21) Appl. No.: 18/884,141

(22) Filed: Sep. 13, 2024

(51) Int. Cl. A61B 5/318; A61B 5/00; A61B 5/0245; A61B 5/26; A61B 5/263; A61B 5/282

(52) U.S. Cl. CPC A61B 5/318; A61B 5/0245; A61B 5/26; A61B 5/263; A61B 5/282; A61B 5/6887; A61B 5/742; A61B 2560/0214; A61B 2560/045; A61B 2560/0468; A61B 2562/166; A61B 2562/185

(57) ABSTRACT

A card capable of measuring ECG signals includes a card body, a control unit, an electronic control unit, a conduction unit, an ECG signal processing unit, a storage unit, a link unit and a light source processing unit. The card allows a person to be tested to touch the conduction unit simultaneously with the fingers of both hands, so that a closed circuit is formed between the heart of the person to be tested, the arms of the person to be tested, the conduction unit and the ECG signal processing unit, so as to carry out the measurement of the heart rhythm, the ECG data generated after measurement is stored in the storage unit, the reading device reads the ECG data through the link unit, relevant signals are generated to the light source processing unit for a light source demonstration during the measurement process and after measurement.

### BACKGROUND OF THE INVENTION

### 1. Field of the Invention

The present disclosure relates to a card capable of measuring ECG signals, and in particular to a card capable of measuring ECG signals that can improve the measurement accuracy, convenience and increase the measurement efficiency.

### 2. Description of the Related Art

Generally, when using a card capable of measuring ECG signals for measurement, because the card usually fails to have a relevant display structure, a person to be tested cannot know the measurement result in real time, nor can he know whether the measurement is successful, resulting in a significant reduction in the accuracy and efficiency of the measurement.

Therefore, the present invention aims to disclose an invention of a card capable of measuring ECG signals, so that it can be used for a light source demonstration, and achieve the effects of improving the measurement accuracy, convenience and increasing the measurement efficiency, thereby effectively improving the drawbacks of the prior art.

### BRIEF SUMMARY OF THE INVENTION

In view of the drawbacks of the prior art, the inventor felt that it was not perfected, and has dedicated to research and development of a card capable of measuring ECG signals, in order to generate relevant light source demonstrations during and after the measurement, so as to improve the measurement accuracy, convenience and increase the measurement efficiency.

To achieve the above object and other objects, the present disclosure provides a card capable of measuring ECG signals, which includes: a card body, a control unit, an electronic control unit, a conduction unit, an ECG signal processing unit, a storage unit, a link unit and a light source processing unit. The card body is provided with a light guide frame at an outer edge; the control unit is disposed in the card body, the control unit serves as the operation, processing, setting and control when measuring the ECG signals; the electronic control unit is disposed at the card body and electrically connected to the control unit, the electronic control unit includes a switch portion, a battery and at least one connector, the switch portion is located on a surface of the card body, the battery is located in the card body and electrically connected to the switch portion and the control unit, the connector is located at least one side of the card body and electrically connected to the battery, the switch portion is turned on to make the battery supply power to the control unit, so that the control unit generates a turn-on signal, or the switch portion is turned off to make the battery power off, so that the control unit generates a turn-off signal; the conduction unit is disposed on the surface of the card body, the conduction unit includes a first stainless steel electrode and a second stainless steel electrode adjacent to each other; the ECG signal processing unit is disposed in the card body and electrically connected the control unit and the conduction unit, and a person to be tested touches the first stainless steel electrode and the second stainless steel electrode simultaneously with the fingers of both hands, so that a closed circuit is formed between the heart of the person to be tested, the arms of the person to be tested, the conduction unit and the ECG signal processing unit to carry out a measurement of heart rhythm, a measurement signal is generated during the measurement, and a measurement success signal and an ECG data are generated, or a measurement failure signal is generated after the measurement; the storage unit is disposed in the card body and electrically connected to the control unit, the storage unit stores at least the ECG data; the link unit is disposed in the card body and electrically connected to the control unit, the link unit may be linked with at least one reading device for the transmission of the ECG data; the light source processing unit is disposed in the card body and electrically connected to the control unit, the light source processing unit includes a light control module and a light emitting module, the light control module is electrically connected to the control unit, the light emitting module is electrically connected to the light control module and corresponds to the light guide frame, the light control module receives the turn-on signal, the turn-off signal, the measurement signal, the measurement success signal or the measurement failure signal, in order to drive the light emitting module to cooperate with the light guide frame for a light source demonstration.

In one embodiment of the present disclosure, the card body includes a light shielding panel, a light shielding backplate and a circuit carrier board, the circuit carrier board is disposed between the light shielding panel and the light shielding backplate, the control unit, the electronic control unit, the conduction unit, the ECG signal processing unit, the storage unit, the link unit and the light source processing unit are disposed on the circuit carrier board.

In one embodiment of the present disclosure, the light control module is provided with a turn-on signal driver, a turn-off signal driver, a measurement signal driver, a measurement success signal driver and a measurement failure signal driver.

In one embodiment of the present disclosure, the turn-on signal driver receives the turn-on signal to drive the light emitting module to cooperate with the light guide frame to carry out the light source demonstration of start-up; the turn-off signal driver receives the turn-off signal to drive the light emitting module to cooperate with the light guide frame to carry out the light source demonstration of shutdown; the measurement signal driver receives the measurement signal to drive the light emitting module to cooperate with the light guide frame to carry out the light source demonstration of tempo of heart rhythm; the measurement success signal driver receives the measurement success signal to drive the light emitting module to cooperate with the light guide frame to carry out the light source demonstration that the measurement is successful; the measurement failure signal driver receives the measurement failure signal to drive the light emitting module to cooperate with the light guide frame to carry out the light source demonstration that the measurement is failed.

In one embodiment of the present disclosure, the light emitting module is a plurality of LED light strips disposed in the light guide frame, or the light emitting module is a plurality of lateral light emitting diodes corresponding to the light guide frame.

Whereby, the card capable of measuring ECG signals of the present disclosure can allow a person to be tested to touch the conduction unit simultaneously with the fingers of both hands, so that a closed circuit is formed between the heart of the person to be tested, the arms of the person to be tested, the conduction unit and the ECG signal processing unit, so as to carry out the measurement of the heart rhythm, and the ECG data generated after measurement is stored in the storage unit, and the reading device reads the ECG data through the link unit, and relevant signals are generated to the light source processing unit for a light source demonstration during the measurement process and after measurement, thereby achieving the effects of improving measurement accuracy, convenience and increasing measurement efficiency.

### BRIEF DESCRIPTION OF THE DRAWINGS

FIG. 1 is a schematic perspective diagram according to a preferable embodiment of the present disclosure.

FIG. 2 is a schematic block diagram according to the preferable embodiment of the present disclosure.

### DETAILED DESCRIPTION OF THE INVENTION

To fully understand the objects, features and functions of the present disclosure, details of the present disclosure are given in specific embodiments with the accompanying drawings below.

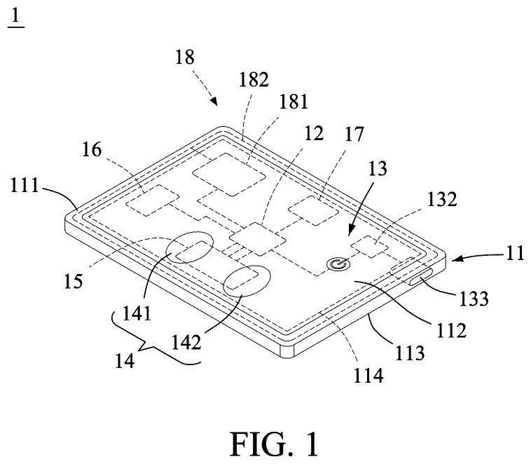

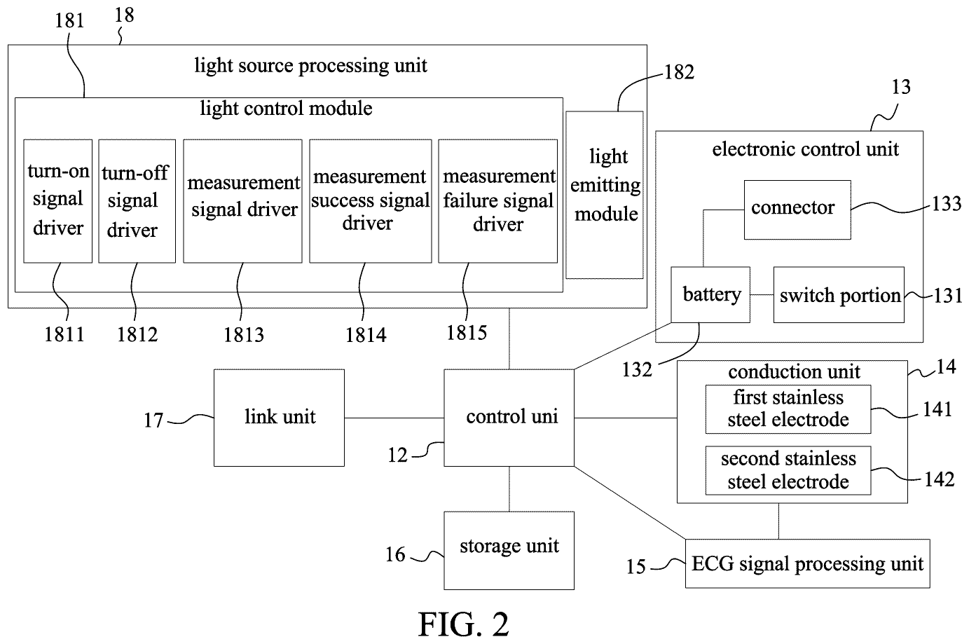

Referring to FIG. 1 and FIG. 2, as shown, the present disclosure provides a card 1 capable of measuring ECG signals, which includes at least a card body 11, a control unit 12, an electronic control unit 13, a conduction unit 14, an ECG signal processing unit 15, a storage unit 16, a link unit 17 and a light source processing unit 18.

The card body 11 is provided with a light guide frame 111 at an outer edge.

The control unit 12 is disposed in the card body 1, and the control unit 12 serves as the operation, processing, setting and control of the electronic control unit 13, the conduction unit 14, the ECG signal processing unit 15, the storage unit 16, the link unit 17 and the light source processing unit 18 when measuring the ECG signals, wherein the control unit 12 may be a central processing unit, a microcontroller, an integrated circuit chip or a logic circuit, and at least may set a single measurement time, a measurement period, etc.

The electronic control unit 13 is disposed at the card body 11 and electrically connected to the control unit 12, the electronic control unit 13 includes a switch portion 131, a battery 132 and at least one connector 133, the switch portion 131 is located on a surface of the card body 11, the battery 132 is located in the card body 11 and electrically connected to the switch portion 131 and the control unit 12, the connector 133 is located at least one side of the card body 11 and electrically connected to the battery 132, the switch portion 131 is turned on to make the battery 132 supply power to the control unit 12, so that the control unit 12 generates a turn on signal, or the switch portion 131 is turned off to make the battery 132 power off, so that the control unit 12 generates a turn-off signal.

The conduction unit 14 is disposed on the surface of the card body 11, the conduction unit 14 includes a first stainless steel electrode 141 and a second stainless steel electrode 142 adjacent to each other.

The ECG signal processing unit 15 is disposed in the card body 11 and electrically connected the control unit 12 and the conduction unit 14, wherein the ECG signal processing unit 15 is a multi-lead cardiac processing unit (ECG), and a person to be tested touches the first stainless steel electrode 141 and the second stainless steel electrode 142 simultaneously with the fingers of both hands, so that a closed circuit is formed between the heart of the person to be tested, the arms of the person to be tested, the conduction unit 14 and the ECG signal processing unit 15 to carry out a measurement of heart rhythm, a measurement signal is generated during the measurement, and a measurement success signal and an ECG data are generated, or a measurement failure signal is generated after the measurement.

The storage unit 16 is disposed in the card body 11 and electrically connected to the control unit 12, the storage unit 16 stores at least the ECG data, and the storage unit 16 may be a flash memory.

The link unit 17 is disposed in the card body 11 and electrically connected to the control unit 12, the link unit 17 may be linked with at least one reading device for the transmission of the ECG data, and the link unit 17 may be a Bluetooth communication interface or a WI-FI communication interface to be used as a wireless transmission with the reading device.

The light source processing unit 18 is disposed in the card body 11 and electrically connected to the control unit 12, the light source processing unit 18 includes a light control module 181 and a light emitting module 182, the light control module 181 is electrically connected to the control unit 12, the light emitting module 182 is electrically connected to the light control module 181 and corresponds to the light guide frame 111, the light control module 181 receives the turn-on signal, the turn-off signal, the measurement signal, the measurement success signal or the measurement failure signal, in order to drive the light emitting module 182 to cooperate with the light guide frame 111 for a light source demonstration, wherein the light emitting module 182 may be a plurality of LED light strips buried in the light guide frame 111, or the light emitting module 182 may be a plurality of lateral light emitting diodes corresponding to the light guide frame 111.

When in use, the switch portion 131 can be pressed to turn on the battery 132 to supply power to the control unit 12, so that the control unit 12 generates the turn-on signal to the light control module 181, the light control module 181 drives the light emitting module 182 according to the turn-on signal, the light emitting module 182 emits a light source (for example, the light source with a blue light signal is emitted when turned on successfully), and the light source cooperates with the light guide frame 111 for a light source demonstration (for example, the light emitting module 182 flashes once corresponding to the light guide frame 111, or the light source of the light emitting module 182 goes around the light guide frame 111 for a circle), so that the light source of the light emitting module 182 cooperates with the light guide frame 111 to inform the person to be tested that the measurement can be carried out.

The person to be tested may touch the first stainless steel electrode 141 and the second stainless steel electrode 142 simultaneously with the fingers (thumbs) of both hands, so that a closed circuit is formed between the heart of the person to be tested, the arms of the person to be tested, the conduction unit 14 and the ECG signal processing unit 15 to measure the heart rhythm, and the measurement signal is generated to the light control module 181 during the measurement, so that the light control module 181 drives the light emitting module 182 according to the measurement signal, and the light emitting module 182 emits a light source (for example, the light source with a blue light signal or a red light signal is emitted during measurement), and the light source cooperates with the light guide frame 111 for a light source demonstration (for example, the light emitting module 182 corresponds to the light guide frame 111 to imitate the tempo of the human heart rhythm and makes it continuously flash), so that the light source of the light emitting module 182 cooperates with the light guide frame 111 to inform the person to be tested that the measurement is in progress with the light source of the tempo of the heart rhythm, and the measurement signal of the ECG signal processing unit 15 at the time of measurement is decoded and encoded by the control unit 12 at the same time, and then the measurement success signal and the ECG data are generated after the measurement; the light control module 181 receives the measurement success signal, and the light control module 181 drives the light emitting module 182 according to the measurement signal, and the light emitting module 182 emits a light source (for example, the light source with a green light signal is emitted when the measurement is successful), and the light source cooperates with the light guide frame 111 for a light source demonstration at the same time (for example, the light emitting module 182 corresponds to the light guide frame 111 and is continuously lit for at least 3 seconds), and the ECG data is stored in the storage unit 16, and the person to be tested has been completed the measurement.

When the measurement is in progress, because the fingers (thumbs) of both hands of the person to be tested move away and fail to touch the first stainless steel electrode 141 and the second stainless steel electrode 142 at the same time, the ECG processing unit 15 generates the measurement failure signal to the light control module 181, the light control module 181 drives the light emitting module 182 according to the measurement signal, the light emitting module 182 emits a light source (for example, the light source with a red light signal is emitted during measurement), and the light source cooperates with the light guide frame 111 for a light source demonstration (for example, the light emitting module 182 corresponds to the light guide frame 111 for a light source demonstration in a fast and continuous flashing mode), so as to prompt the person to be tested that the measurement fails, and let the person to be tested restart and retest.

When the measurement is completed and not in use, the switch portion 131 can be pressed to turn off and the battery 132 is powered off, so that the control unit 12 generates the turn-off signal to the light control module 181, the light control module 181 drives the light emitting module 182 according to the turn-on signal, the light emitting module 182 emits a light source (for example, the light source with a red light signal is emitted before turning off), and the light source cooperates with the light guide frame 111 for a light source demonstration (for example, the light emitting module 182 flashes once corresponding to the light guide frame 111, or the light source of the light emitting module 182 goes around the light guide frame 111 for a circle), so that the light source of the light emitting module 182 cooperates with the light guide frame 111 to inform the person to be tested that the measurement has been shut down.

When the ECG data stored in the storage unit 16 is to be read, the reading device (e.g., a computer, a medical computer or a card reading device) is linked to the link unit 17 in a start-up state, and the ECG data in the storage unit 16 is transmitted to the reading device by means of wireless transmission for subsequent interpretation.

Thus, the card capable of measuring ECG signals of the present disclosure can allow the person to be tested to touch the conduction unit 14 simultaneously with the fingers of both hands, so that a closed circuit is formed between the heart of the person to be tested, the arms of the person to be tested, the conduction unit 14 and the ECG signal processing unit 15, so as to carry out the measurement of the heart rhythm, and the ECG data generated after measurement is stored in the storage unit 16, and the reading device reads the ECG data through the link unit 17, and relevant signals are generated to the light source processing unit 18 for a light source demonstration during the measurement process and after measurement, thereby achieving the effects of improving measurement accuracy, convenience and increasing measurement efficiency.

In addition to the above embodiment, in one embodiment of the present disclosure, it is different from the above embodiment in that the card body 11 includes a light shielding panel 112, a light shielding backplate 113 and a circuit carrier board 114, the circuit carrier board 114 is disposed between the light shielding panel 112 and the light shielding backplate 113, the control unit 12, the electronic control unit 13, the conduction unit 14, the ECG signal processing unit 15, the storage unit 16, the link unit 17 and the light source processing unit 18 are disposed on the circuit carrier board 114, wherein the light shielding panel 112 and the light shielding backplate 113 may be made of polyethylene terephthalate (PET).

Based on the above embodiment, during production, a full-page pattern may be printed on the light shielding panel 112 and the light shielding back plate 113, and then the light guide frame 111 is added, or the pattern design of the position of the light guide frame 111 may be reserved, so as to increase the aesthetics of the card body 11 and obtain a preferable light source effect, and be used as the protection of the circuit carrier board 114, the control unit 12, the electronic control unit 13, the conduction unit 14, the ECG signal processing unit 15, the storage unit 16, the link unit 17 and the light source processing unit 18, and greatly reduce the complexity and precision of production, and maintain the stability of the control unit 12, the electronic control unit 13, the conduction unit 14, the ECG signal processing unit 15, the storage unit 16, the link unit 17 and the light source processing unit 18 when operating, and achieve improving the yield rate during production.

In addition, the shading panel 112, the shading backplate 113 and the circuit carrier board 114 may be combined by cold lamination technology; thus, the light guide frame 111, the shading panel 112 and the shading backplate 113 can avoid warping, the deformation of the flexible electronic control carrier board 14 can be avoided, and the influence of the electrical characteristics of the control unit 12, the electronic control unit 13, the conduction unit 14, the ECG signal processing unit 15, the storage unit 16, the link unit 17 and the light source processing unit 18 can be avoided, so that the card 1 capable of measuring ECG signals reaches structural stability and maintains stability during use.

In addition to the above embodiments, in one embodiments of the present disclosure, it is different from the above embodiments in that the battery 132 may be a primary battery or a secondary battery, and the connector 133 may be a socket connector (e.g., a Type-C connector); when the battery 132 is a secondary battery, the connector 133 can be used to cooperate with the required transmission line or take utility power or equipment power to charge the battery 132; in addition, the electronic control unit 13 may also be provided with a wireless charging module according to demand, and the wireless charging module is electrically connected to the battery 132 (secondary battery), so that the card 1 capable of measuring ECG signals is directly placed on a wireless charging pad during charging, so as to use the wireless charging module to match with the wireless charging pad for wirelessly charging the battery 132.

In addition, in addition to being used with the required transmission line for charging, the connector 133 can also be connected to the reading device (such as a computer, a medical computer or a card reading device) through the transmission line, so as to read the ECG data stored in the storage unit 16 by means of wired transmission for subsequent interpretation.

In addition to the above embodiments, in one embodiments of the present disclosure, it is different from the above embodiments in that the light control module 181 is provided with a turn-on signal driver 1811, a turn-off signal driver 1812, a measurement signal driver 1813, a measurement success signal driver 1814 and a measurement failure signal driver 1815.

Based on the above embodiment, the light control module 181 can be used to receive the turn-on signal, the turn-off signal, the measurement signal, the measurement success signal or the measurement failure signal and then drive the light emitting module 182 to emit an appropriate light source; in the embodiment of the light control module 181, the turn-on signal driver 1811 can receive the turn-on signal to drive the light emitting module 182 to cooperate with the light guide frame 111 to carry out the light source demonstration of the above-mentioned start-up; the turn-off signal driver 1812 receives the turn-off signal to drive the light emitting module 182 to cooperate with the light guide frame 111 to carry out the light source demonstration of the shutdown; the measurement signal driver 1813 receives the measurement signal to drive the light emitting module 182 to cooperate with the light guide frame 111 to carry out the light source demonstration of tempo of heart rhythm; the measurement success signal driver 1814 receives the measurement success signal to drive the light emitting module 182 to cooperate with the light guide frame 111 to carry out the light source demonstration that the measurement is successful; the measurement failure signal driver 1815 receives the measurement failure signal to drive the light emitting module 182 to cooperate with the light guide frame 111 to carry out the light source demonstration that the measurement is failed. Thus, the relevant signals can be generated to the light source processing unit 18 for a light source demonstration during the measurement process and after measurement, thereby achieving the effects of improving measurement accuracy, convenience and increasing measurement efficiency.

While the invention has been described by way of example and in terms of the embodiments, a person skilled in the art should understood that the embodiments are for illustrating the present invention and are not to be construed as limitations to the scope of the present invention. It should be noted that, equivalent modifications and substitutions made to the embodiments are to be encompassed within the scope of the present invention. Therefore, the protection scope of the present invention is to be accorded with the appended claims.

What is claimed is:

1. A card capable of measuring ECG signals, comprising:
a card body, provided with a light guide frame at an outer edge;
a control unit, disposed in the card body, the control unit serves as the operation, processing, setting and control when measuring the ECG signals;
an electronic control unit, disposed at the card body and electrically connected to the control unit, the electronic control unit comprises a switch portion, a battery and at least one connector, the switch portion is located on a surface of the card body, the battery is located in the card body and electrically connected to the switch portion and the control unit, the connector is located at least one side of the card body and electrically connected to the battery, the switch portion is turned on to make the battery supply power to the control unit, so that the control unit generates a turn-on signal, or the switch portion is turned off to make the battery power off, so that the control unit generates a turn-off signal;
a conduction unit, disposed on the surface of the card body, the conduction unit comprises a first stainless steel electrode and a second stainless steel electrode adjacent to each other;
an ECG signal processing unit, disposed in the card body and electrically connected the control unit and the conduction unit, a person to be tested touches the first stainless steel electrode and the second stainless steel electrode simultaneously with the fingers of both hands, so that a closed circuit is formed between the heart of the person to be tested, the arms of the person to be tested, the conduction unit and the ECG signal processing unit to carry out a measurement of heart rhythm, a measurement signal is generated during the measurement, and a measurement success signal and an ECG data are generated, or a measurement failure signal is generated after the measurement;
a storage unit, disposed in the card body and electrically connected to the control unit, the storage unit stores at least the ECG data;
a link unit, disposed in the card body and electrically connected to the control unit, the link unit can be linked with at least one reading device for the transmission of the ECG data; and
a light source processing unit, disposed in the card body and electrically connected to the control unit, the light source processing unit comprises a light control module and a light emitting module, the light control module is electrically connected to the control unit, the light emitting module is electrically connected to the light control module and corresponds to the light guide frame, the light control module receives the turn-on signal, the turn-off signal, the measurement signal, the measurement success signal or the measurement failure signal, in order to drive the light emitting module to cooperate with the light guide frame for a light source demonstration.

2. The card capable of measuring ECG signals of claim 1, wherein the card body comprises a light shielding panel, a light shielding backplate and a circuit carrier board, the circuit carrier board is disposed between the light shielding panel and the light shielding backplate, the control unit, the electronic control unit, the conduction unit, the ECG signal processing unit, the storage unit, the link unit and the light source processing unit are disposed on the circuit carrier board.

3. The card capable of measuring ECG signals of claim 1, wherein the light control module is provided with a turn-on signal driver, a turn-off signal driver, a measurement signal driver, a measurement success signal driver and a measurement failure signal driver.

4. The card capable of measuring ECG signals of claim 3, wherein the turn-on signal driver receives the turn-on signal to drive the light emitting module to cooperate with the light guide frame to carry out the light source demonstration of start-up;
the turn-off signal driver receives the turn-off signal to drive the light emitting module to cooperate with the light guide frame to carry out the light source demonstration of shutdown; the measurement signal driver receives the measurement signal to drive the light emitting module to cooperate with the light guide frame to carry out the light source demonstration of tempo of heart rhythm; the measurement success signal driver receives the measurement success signal to drive the light emitting module to cooperate with the light guide frame to carry out the light source demonstration that the measurement is successful; the measurement failure signal driver receives the measurement failure signal to drive the light emitting module to cooperate with the light guide frame to carry out the light source demonstration that the measurement is failed.

5. The card capable of measuring ECG signals of claim 1, wherein the light emitting module is a plurality of LED light strips disposed in the light guide frame, or the light emitting module is a plurality of lateral light emitting diodes corresponding to the light guide frame.
